(19) United States
(12) Patent Application Publication
(10) Pub. No.: US 2007/0166289 A1
(43) Pub. Date: Jul. 19, 2007

(54) AUTOLOGOUS HUMAN DNA GRAFTING - ANTI- & REVERSE AGING PROCESS, METHOD

(76) Inventors: Alecia A. Hathaway, Fort Worth, TX (US); William C. Orr, Denver, CO (US)

Correspondence Address: HOGAN & HARTSON LLP, ONE TABOR CENTER, SUITE 1500, 1200 SEVENTEENTH ST, DENVER, CO, 80202, US

(21) Appl. No.: 11/531,220

(22) Filed: Sep. 12, 2006

Related U.S. Application Data
(60) Provisional application No. 60/716,207, filed on Sep. 12, 2005.

(51) Int. Cl. A61K 35/12; C12N 5/08; C12N 5/04

(52) U.S. Cl. 424/93.7; 435/372; 435/419

(57) ABSTRACT

Anti-aging process and method for delivering younger human DNA to tissues, through the periodic infusion (auto-transfusion) of previously harvested autologous younger stem cells, resulting in re-establishment of an earlier relative biological clock set-point, with respect to the number of cell generations-divisions.

### RELATED APPLICATIONS

The present application claims priority of U.S. Provisional Application No. 60/716,207 filed Sep. 12, 2005, which is incorporated herein in its entirety by this reference.

### SUMMARY OF INVENTION

This invention resides in periodic/regular collection of stem cells from a living being at regular intervals predominately during successive stages of their youth beginning in infancy and continuing throughout their life; dedicated for the sole use of the donor individual; in sufficient quantities; then appropriately and securely stored, batched in an environment to prevent their aging, cataloged by collection dates, etc, and entered into a data base and backed-up hundreds of miles elsewhere; then later infused back into the same being from whom the stem cells were harvested (the donor-recipient or auto-transfusion) in ascending order of collection batches (or under other design) at opportune times (to be determine through mathematical regression models) in successive regular stages throughout the being's entire adulthood life.

The practice of this invention contemplates a wide range of mammalian beings but more preferably human beings.

The invention in time results in the infusion of stem cells back into the donor-recipient being, which have a biological age less than the biological age of the recipient. In time there is an even greater age difference when comparing the infusion's average biological DNA age to the recipient's chronological age. So, there are at least 2 age differentials with respect to the infused stem cells, biological to biological and biological to chronological.

### DETAILED DESCRIPTION OF THE INVENTION

Applicant's invention is predicated on 5 fundamental elements/steps:
1. Periodic collection of stem cell material from a donor, whereby the mass of all collected samples has an average biological age ranging from birth to 70 years +/−of age, with preferred biological age ranging from birth to under 50, and a more preferred range from birth to under 30.
2. Concentration/sorting this stem cell material such that it can be effectively stored for very extended times, absent appreciable aging, and then later reused as a live organic medium.
3. Storing the stem cell medium, whereby said samples do not appreciably age and can be infused into a mammalian being up to decades or centuries later.
4. Periodic infusion into the donor after achieving a minimum age on a global basis, anticipated to be over their entire lifetime, wherein
5. The continuation of this periodic stem cell collection and stem cell infusion regime results in an average chronological and/or biological age difference of infused DNA (that is appreciably—at least 5 years younger) compared the chronological age of the donor.

Naturally, there are many derivatives and variations of this invention, which embody the aforementioned elements. These are contemplated herein,

In sum, this invention embodies a elegant and unique delivery system to replace the body's older cell structures throughout the entire body with younger more vital cells, resulting in a transformation of tissue structures and organs (on a global basis) to a younger version.

Mammalian cells are pre-programmed, through their genome (DNA mechanism) so as to undergo a finite number of cellular replications—divisions. As the individual cells (which define the total tissue structure) progress along their continuum of number of cellular divisions, their rate of replication slows. Eventually, signs of aging become apparent, as damaged and worn tissue cells are not timely replaced. Cells become worn, exhausted, damaged from environmental insults, and deprived of necessary co-factors and interactive influences from other failing bodily endocrine and physiologic functions.

While research efforts are and have been underway to intervene in the aging process, none propose to simply replace the entire body's cells with younger, fresh cells through a self-adaptive (autologous) grafting process, employing the very body and DNA of the recipient.

Human stems cells contain the body's complete genetic information, factors, determinants and capacity for generation of differentiated tissues throughout the entire body of a person into specific end-organ and support tissues such as bone, nerves, muscle, heart, liver, brain, endocrine organs, skin etc. Human stems cell can be collected, stored and re-infused at a later date into the bloodstream of the same person from which they were harvested at an earlier time in his/her life. They will disseminate throughout the entire body, lodging in all tissues. Interacting with factors and determinants specific to differentiated ‘resident’ tissue cells (already evolved to a specific and distinguishable type of tissue such as bone or muscle, etc.), the stem cells will be induced through interactions with the adopted tissues to ‘differentiate’ or evolved into the respective distinguishable tissues in which they find residence forming a graft and transient chimera of older and younger DNA-containing cells.

Possessing younger DNA, vital enzymes and co-factors, these younger stem cells will divide at faster rates than their adopted perspective resident mature host tissue cells and then mature into new and younger host cells, “replacing” the older resident cells, eventually resulting in an essentially homogeneous cellular composition with respect to the age of the cells' DNA (thus establishing a younger total body).

Infusion with sufficient quantities of vital stem cells over regular, adequate and periodic time-frames would result in successive, but eventually diminishing replacement of aging tissue. Because, it is not known what tissues may have an inherent resistance to ‘replacement’; it is not known what quantities of stem cells would actually be needed and what time period it would take for total tissue replacement with the younger newly differentiated cells; and while it might be presumed based upon natural statistical truths, it is not known if there is a dose-time response curve (more stem cells infused—the faster they take over by replacing the older tissue cells). In addition, one's supply of viable stem cells would eventually be exhausted, as would their and perhaps society's will and resources. An endless supply of one's younger stem cells could not be obtained to be indefinitely accessed by a person later in his/her life.

Prior art research focuses primarily on utilizing human DNA that is obtained in stem cells harvested from donors other than the intended recipient (non-autologous, i.e. embryonic or fetal stem cells). Still other prior art explores autologous DNA obtained from stem cells collected from the donor in real-time or relatively current to the recipient's age and treatment period. Other art discloses both types involve “injecting” whole stem cells (whether autologous or not) into target tissue to promote repair, such as in spinal cord injury or damaged heart.

It is not known or disclosed in the prior art, whether repair occurs due to unique stimulatory factors inherent to stem cells that are exerted upon the resident differentiated damaged tissues or if repair results from the stem cells differentiating themselves into undamaged tissue. To date, the vast focus of autologous stem cell repair employs enucleating the stem cell and transferring this DNA into enucleated cells of the host's tissue to be repaired. This process of enucleating and transferring is mechanically performed.

The applicants instant invention distinguishes over the prior art because it is predicated upon cells being transferred globally “throughout the entire body,” neither involving injecting cells or their contents or any portion thereof. Nor does it involve a directed transferring of the contents of cells (i.e. DNA) into either tissues or into enucleated tissue cells. This invention distinguishes because it requires pre-planned protocols, pre-positioned resources for an intended recipient prior to or soon thereafter their birth, and deliberate and periodic collection, storing and deliberate, and periodic autologous whole stem cell infusions (auto-transfusions) on a global whole body basis.

### EXAMPLE 1

A process or method employing: a) periodicity of infusions offset by the deliberate and regular periodic collections of a person's stem cells, under a protocol that would follow a staggered or stair-stepped ascending schedule—the earliest collected stem cells are utilized first; b) where those collected in the early years (such as in the first decade) would be used for the first infusions in the 1^{st}, 2^{nd}, 3^{rd}, 4^{th} and later decades (depending on supply quantities), and where stem cells collected in the 1^{st}, 2^{nd}, 3^{rd}, 4^{th} and later decades would be used in the 5^{th} and 6^{th} decades, and so on—with obvious adjustment as time, supply and need would dictate.

### EXAMPLE 2

An anti-aging method or process: a) tanking periodic collection of stem cells from a donor from his birth to his age 500 years or less (until some determinate period prior to this death); b) sorting and concentrating these stem cells in a collection of blood sera contents, such that they would represent a plurality of blood product constituents for use at a future date; c) storing the donor-recipient stem cells in sterile conditions in non-breachable containers under sub-zero temperature sufficient to insure future use; d) thawing a minor portion of said stored stem cells after a period of 1 to 500 years, depending upon the protocol and program of the recipient; e) then making periodic auto-transfusions of the stored stem cells, which would be thawed for use, back into same donor (recipient) starting after a responsible age (likely to be after donor's chronological age 10 to 30 years); f) conducting this protocol in such a manner that the infusion of stem cell would result in an integration of biologically younger stem cells into recipient's tissues throughout his/her entire body

It is worth bearing in mind that each successive decade of life will actually be characterized by a younger specimen, resulting from prior infusions of younger stem cells that have consequently replaced the older tissue cells. New supplies of younger stem cells will be produced in the younger person to be used and stored for later use despite their chronological age.

In actuality, the use of Applicants' invention may be elected at any point in a person's life—realizing that optimal benefit of extending youth is achieved using the youngest possible DNA-containing stem cells—(with at least an age differential of 10 years and ideally obtained in the first decade of life). This is the biggest challenge, however—obtaining adequate quantities of stem cells during one's 1^{st} decade to survive years of storage for use later in life. While focus on harvesting stem cells during childhood is emphasized, regular collections should continue, throughout the span of a person's life, in order to assure supplies later on and continue the infusions and anti-aging benefit.

Periodic and regular collection makes it possible to store the necessary minimum amounts of autologous whole stem cell (DNA) to practice this invention, and is an essential element.

As contemplated herein periodic collection may begin a birth and continue until prior to the death of the individual. It is anticipated that this invention will practiced over normal life times, which there under will range from birth to age 100, 150, 200, 300, 400, 500, or more years. The ultimate success of the invention has yet to be determined. It is conceivable human life could be extended to age 1000 years.

Periodic stem cell collection will depend upon the specific program results desired, age at which donor starts the regime and other factors. However, expected collections will start at birth and last a life time. The contemplated collection of stem cells is by harvesting stem cells, through sterile mass blood collection and/or cellular dialysis or continuous circulating cell screening techniques, or others contemplated.

It is fully expected that processes/devices and/or methods, including protector, promoter, enhancing, inducing, suppressing, and/or inhibiting, and/or other agents, known and/or which will be developed specific for: 1) harvesting of targeted stem cells from the vascular compartment, bone marrow, and the like, 2) screening and concentration, and the like, and 3) their subsequent placement back into donor/recipient's tissues at later date, will be employed.

It is contemplated that these processes/devices and/or methods may or may not be concurrently used during collection and/or infusion, and may be used at some time before and/or after said collection and/or infusion.

These processes/devices and/or methods are incorporated into the claims of this invention.

The continuous collection of stem cells is of paramount importance. It is desirable under this invention to obtain sufficient quantities with a goal of no less than 50 to 500 grams per year, more preferably 100 to 2000 grams. Lesser amounts of 1 to 20 grams per year would also be acceptable. Ideally, collection will need to target as much as possible in the earliest years.

A preferred collection period would include collecting 5 grams to 1000 grams of packed stem cells on a frequently of 1 to every 6 months. Another desirable periodic collection frequency includes at least once every 12 months. Other frequencies include once every 1 to 5 years. Yet another example would be collections of no less than once a year for a donor's age 1 & 2, twice a year for ages 3 to 5, three times a year ages 6 through 15, four times a year 16 years or older. Other periods/frequencies are contemplated. Even so, periodic collection as contemplated in the claims hereto, include periods that are irregular in length and random in nature.

Thus, a principal embodiment of this invention is dedicated periodic collection, so that there exists a major amount of youthful stem cells stored by an early age, sufficient to accomplish the life time regime of this invention.

An objective of this invention is to have collected at least 0.01 to 3 kilograms of concentrated stem cell material by a donor's chronological age of 20 to 30 years. Acceptable amounts would be 0.02 to 0.5 kilograms. Similar amounts at chronological age of 30 to 40 years would be acceptable. Concentrated stem cell material of at least 0.2 to 1.0 kilograms would however be more desirable. Concentrations ranging from about 1 kilogram to 6 kilograms and from 1.5 kilograms to 5 kilograms by age 30 would be more desirable. Concentration ranges outside these ranges are expressly contemplated in the practice of this invention.

The invention further distinguishes in that it requires a life-long commitment of the recipient and is for the sole use of the donor-recipient.

The current art employs the technique of auto-transfusions on an as needed basis for whole blood or blood product transfusions, in response to emergency and life threatening incidents or anticipated surgeries for patients with rare blood types, such as AB (−). Collection and storage of blood is sporadic and not conducted over a person's lifetime. The intended product of collection are red blood cells predominately with a smaller percentage of white cells and blood clotting components, as is the case industry wide in blood donation.

Applicants' invention is distinguished because its autologous blood components are concentrated to reflect a plurality of stem cells for a global infusion of younger DNA into the human body. Thus, it is a completely different product than disclosed in the prior art.

### EXAMPLE 3

A composition containing DNA in a stem cell carrier, where the DNA is can be delivered in sufficient useable quantity to an autologous donor/recipient to regenerate his existing cell structure on a global body basis.

The above composition is collected according to a predetermined life-time regular regimen with an emphasis on the person's 1st and 2^{nd} decades, with stem cell auto-transfusions performed on a predetermined periodicity throughout a person's life, distinguished from being transfused on a stand-by basis, for example, in response to an emergency or an anticipated surgery;

It is further distinguished because it does not target site specific tissue repairs or regeneration. Instead it is an anti-aging mechanism that performs by replacing older tissue cells throughout the “entire” body.

Applicant's invention employs a technique whereby ‘whole’ stem cells are globally ‘infused’ via the blood stream, disseminating throughout the whole of the body, rather than site specific injections. The invention, thus, capitalizes on the body's natural design and physiologic mechanisms, which permit the receiving and lodging of the newly introduced stem cells into the tissue; integration, feedback and stimulation that results in differentiation and maturation of the stem cells into the adopted resident tissues. It distinguishes in that no other interventions, chemically or mechanically are performed or induced to affect this result, unlike the current art. And the current art exploring anti-aging modalities and technology employs no similar or obvious modal.

The prior art also utilizes stem cells or their components for the purpose of growing or generating tissues in a laboratory setting for later use in repairing or regenerating specific target tissues within the body. Other prior art focuses on specific techniques exploring sub-components of stem cells and their genetic material in an attempt to control the determinants that regulate growth and cellular behavior.

Instant invention intervenes with the aging process and operates to replace damaged tissue globally, through the use of stem cells and/or their components. It simply recruits the body's own inherent and complex processes to reset its biological clock, through saving its own natural resources (younger stem cells) without complex interference to natural body processes or regulation mechanisms; and re-introduces these said resources to the whole body, through a simply infusion into the blood stream. Claimed invention globally disseminates stem cells that carry younger autologous DNA throughout the entire body that will eventually “replace” the existing older tissue cells.

The body does not regress, but is transformed to a younger more vital stage. Acquired attributes would be transferred by the body's inherent redundancies in biological systems. For example, we know that memory and other ‘information’ is transferred to new cells from older ones.

Thus, the claimed invention extends youth.

There will be eventual genetic fade. That is, each successive collection point will yield stem cells a little bit older than the proceeding. Thus, a person will still age, but theoretically much more slowly. However, the person's biological age and chronological age will widen. The biological age will advance, but at a slower rate than the person's actual chronological age. It will take much longer to reach the end of the reverse moving platform. But, it will eventually be reached.

Extending youth is to be distinguished from extending longevity—as this is occurring, through enhancements in medical services and technology; public health and preventive medicine via vaccines, medications, early detection and intervention of diseases, enhanced nutrition, reduced exposure to environmental insults etc., healthful living choices and habits; and through improved environmental engineering.

Applicant's invention posses no risk to the individual from rejection reaction, nor requires use of medications to prevent rejection reactions, delicate or risky invasive procedures or toxic effects, which is a distinguishing feature.

The following is excluded from risk exception, such as idiosyncratic reactions or anaphylaxis to any carrier solution or residue, equipment or its materials used to collect or re-infuse the stem cells or to unforeseen events due to poor technique, sanitation, inventory error or other errors—as its potential occurrence is not unique.

The economics of longer life-spans would be offset by cost-shifting of expensive sick care consumptive costs and the long-term financial and social commitment of sustaining lives suffering with chronic illnesses, as people would live longer—healthier!

1. An anti-aging method comprising:
a. periodic collection of stem cells from donor from birth to age 500 years;
b. sorting and concentrating said stem cells of step 1 in the collected sera contents, such that they represent a plurality of blood product constituents;
c. storing said donor-recipient stem cells of step 2 in sterile conditions in non-breachable containers under sub-zero temperature (equal or less than 0° F.);
d. thawing a minor portion of said stored stem cells of step 3 after a period of 1 to 500 years;
e. periodic auto-transfusions of said thawed stem cells back into same donor (recipient) starting after said donor's chronological age 10; and
f. whereby said infusion results in an integration of biologically younger stem cells into recipient tissues throughout his/her body.

2. (canceled)

3. (canceled)

4. The method of claim 1 to 3, wherein collection of said stem cells is by means specific for harvesting targeted cells from the vascular compartment.

5. The method of claim 1 and 2, wherein collection of said stem cells is by concentration means specific for harvesting targeted cells from bone marrow.

6. The method of claim 1, wherein concentration of said stem cells, is by means sufficient to result in a plurality of stem cells, which are in combination with blood product constituents.

7. The method of claim 1, wherein collection results in storage of from 50 to 1000 grams of stem cells or material containing a plurality to a majority of said stem cells.

8. (canceled)

9. (canceled)

10. (canceled)

11. The method of claim 1, where said mass is collected by donor's chronological age of 10.

12. The method of claim 1, where said mass is collected by donor's chronological age of 20.

13. (canceled)

14. (canceled)

15. (canceled)

16. The method of claim 1, wherein periodic collection occurs one (1) to twelve (12) times annually.

17. (canceled)

18. The method of claim 1, wherein periodic collection is no more frequently than every 6 months.

19. (canceled)

20. (canceled)

21. (canceled)

22. (canceled)

23. (canceled)

24. (canceled)

25. The method of claim 1, including means to increase the quantity, quality and accessibility of stem cells within the circulation.

26. (canceled)

27. (canceled)

28. The method of claim 1, wherein the minimum mass of stem cells collected is equivalent to 10% to 100% of a standard unit of red blood cells (no less than 25 grams) standard in the blood banking industry.

29. The method of claim 1, wherein said storing of said donor-recipient stem cells is under sterile conditions in non-breachable containers at temperature below 0° F.

30. (canceled)

31. (canceled)

32. (canceled)

33. (canceled)

34. The method of claim 29, wherein said temperature is a cryogenic freezing temperature sufficient to insure long term storage of said stem cells.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. The method of claim 1, wherein said infusion incorporates means for selecting stem cells for maximizing anti-aging response.

48. (canceled)

49. (canceled)

50. The method of claim 1, where said infusion is by single or multiple access port of entry into the blood stream, whereby said infused stem cells find their way to all body tissue sites.

51. The method of claim 50, wherein said stem cells, lodge and become integrated into said donor's cell structure (integrated component).

52. The method of claim 51, wherein said integration is by self means.

53. The method of claim 1, wherein said infusion is characterized as being global, whereby autologous stem cells are delivered to all sites, throughout the recipient body, wherein
a. biologically younger stem cells are integrated into recipient's tissues throughout his/her entire body;
b. a transient chimera is formed, characterized by the collective tissue composition of the recipient current chronologically older DNA-containing cells and biologically younger DNA-containing infused autologous stem cells, which were collected and stored years earlier; and
c. the age differential of cells comprising the resulting transient tissue chimera is 5 years or greater.

54. (canceled)

55. The method of claim 1, whereby said infusions begin no later than in said donor's 1 st decade of chronological life.

56. (canceled)

57. (canceled)

58. (canceled)

59. (canceled)

60. (canceled)

61. The method of claim 1, whereby said infusions are made at intervals ranging from once every month to once every 5 years.

62. The method of claim 1, wherein said infusion into said donor is under a periodicity of no less than once every 5-years.

63. The method of claim 1, wherein said infusion into said donor of is under a periodicity of no less than at 10-year intervals.

64. (canceled)

65. (canceled)

66. (canceled)

67. The method claim 1 wherein infusion continues until at least chronological age of 100 years.

68. The method of claims claim 1 wherein infusion continues until at least chronological age of 200 years.

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. The method of claims 71 to 80, wherein the difference between resulting average biological age of the recipient's transformed body following infusions of DNA-containing stem cells is at least 5 years younger than the recipient's chronological age.

82. The method of claim 81 wherein the difference is at least 10 years.

83. The method of claim 81 wherein the difference is at least 20 years.

84. (canceled)

85. (canceled)

86. (canceled)

87. (canceled)

88. (canceled)

89. (canceled)

90. (canceled)

91. (canceled)

92. (canceled)

93. The method of claim 1, wherein the activity resulting from said infusion into said donor results in global cellar replacement.

94. The method of claim 1 wherein the infusions are performed on a predetermined periodicity.
